(19) United States
(12) Patent Application Publication
(10) Pub. No.: US 2010/0317729 A1
(43) Pub. Date: Dec. 16, 2010

(54) NEW PHARMACEUTICAL FORMULATION COMPRISING CANNABIDIOL AND TETRAHYDROCANNABIDIVARIN

(75) Inventors: Geoffrey Guy, Dorchester (GB); Brian Anthony Whittle, Hornsea (GB); Roger Pertwee, Aberdeen (GB)

Correspondence Address: WOLF GREENFIELD & SACKS, P.C., 600 ATLANTIC AVENUE, BOSTON, MA, 02210-2206, US

(73) Assignee: GW Pharma Limited, Salisbury, Wiltshire (GB)

(21) Appl. No.: 12/667,555

(22) PCT Filed: Jul. 4, 2008
(86) PCT No.: PCT/GB08/02315

§ 371 Date: Aug. 5, 2010

(30) Foreign Application Priority Data
Jul. 6, 2007 (GB) 0713175.8

(51) Int. Cl. A61K 31/352; A61K 31/05; A61P 3/04; A61P 25/18; A61P 19/10; A61P 25/32; A61P 25/34; A61P 25/30; A61P 29/00

(52) U.S. Cl. 514/454; 514/729

(57) ABSTRACT

The present invention relates to a novel pharmaceutical formulation comprising a ratioed mix of: (i) one or more compounds that acts as an inverse agonist of the CB1 and/or CB2 receptor; and (ii) one or more compounds that acts as a neutral antagonist of the CB₁ and/or CB₂ receptor. Preferably both the inverse agonist of the CB₁ and/or CB₂ receptor and the neutral antagonist of the CB₁ and/or CB₂ receptor are cannabinoids. Preferably the cannabinoids are tetrahydrocannabidivarin (THCV) and cannabidiol (CBD).

### FIELD OF THE INVENTION

The present invention relates to a novel pharmaceutical formulation comprising a ratioed mix of: (i) one or more compounds that acts as an inverse agonist of the CB₁ and/or CB₂ receptor; and (ii) one or more compounds that acts as a neutral antagonist of the CB₁ and/or CB₂ receptor. Preferably both the inverse agonist of the CB₁ and/or CB₂ receptor and the neutral antagonist of the CB₁ and/or CB₂ receptor are cannabinoids. Preferably the cannabinoids are tetrahydrocannabidivarin (THCV) and cannabidiol (CBD).

### BACKGROUND DESCRIPTION

Cannabinoids are a group of chemicals known to activate cannabinoid receptors in cells. These chemicals, which are found in cannabis plants, are also produced endogenously in humans and other animals, and are termed endocannabinoids. Synthetic cannabinoids are manmade chemicals with the same structure as plant cannabinoids or endocannabinoids.

Cannabinoids are generally known to be cannabinoid receptor agonists. When a cannabinoid receptor agonist binds to a cannabinoid receptor a response is triggered. This response is known as a signalling pathway.

Compounds which are known to bind to the CB₁ cannabinoid receptor include delta-9-tetrahydrocannabinol (THC), R-(+)-WIN55212 and anandamide. These compounds are as such described as CB₁ agonists as when they bind to the CB₁ receptor a specific response is produced.

Agonism at a receptor will often lead to an active response by the cell. Many disease states result from the overactive or overabundant effects of agonists at their receptors.

Cannabinoid receptors are known to be constitutively active. This means that the receptors undergo some degree of coupling to their signalling pathways even in the absence of an agonist. As such they exhibit a background tone.

In the presence of an agonist this background tone is increased. This can cause an intensification of a disease state that has resulted from the active response of the cell.

Research into compounds that are able to oppose the ability of such agonists has led to the discovery of compounds that act as cannabinoid receptor antagonists.

A neutral antagonist is a compound that will bind to the receptor but will lack any efficacy as a receptor agonist. Such a neutral antagonist will compete with agonists for its receptor and once bound will not result in any active response. In constitutively active receptors the background tone remains unaffected.

An inverse agonist will also bind to its receptor and will lack any efficacy as a receptor agonist. Once an inverse agonist is bound to a receptor it is able to produce an opposite effect of the active response.

Therefore in constitutively active receptors an inverse agonist is able to either partially or completely switch off the background tone.

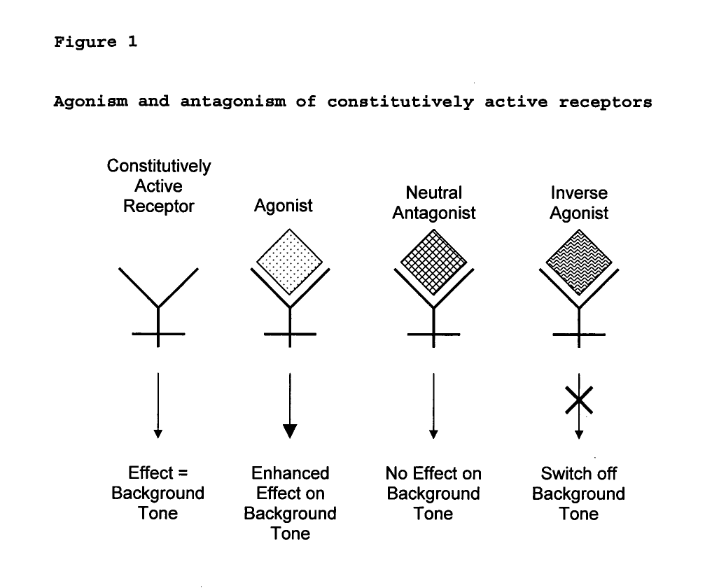

The way in which constitutively active receptors work in the presence of agonists and different types of receptor antagonists is shown in FIG. 1.

The ability of a compound to have antagonistic properties at a constitutively active receptor may be extremely beneficial in the treatment of diseases where a change in the background tone of a cell is the cause of the disease state.

Examples of diseases and conditions that are the result of the background tone of constitutively active cannabinoid receptors include but are not limited to obesity, schizophrenia, epilepsy, cognitive disorders such as Alzheimer's disease, bone disorders such as osteoporosis, bulimia, obesity associated with type II diabetes (non-insulin dependant diabetes), the treatment of drug, alcohol and nicotine abuse or dependency and inflammatory disorders (Pertwee, R. G., 2000).

There is evidence that the endogenous CB₁ agonist, anandamide, is released in the brain to mediate processes such as feeding and appetite (Di Marzo et al., 2001). This raises the possibility that a CB₁ receptor antagonist could be effective in the clinic as an appetite suppressant.

One such cannabinoid receptor antagonist is SR141716A. The use of this compound in the regulation of appetite has been described by Maruani and Soubrie in U.S. Pat. No. 6,444,474 and EP0969835.

The compound SR141716A is a synthetic compound and as such its long-term effects cannot be completely quantified by clinical trials. It is not known how a synthetic compound such as this will interfere with the cannabinoid receptors on a very long-term basis (it is likely from data accumulated in a clinical study with SR141716A that appetite suppressant treatments will have to be chronic). The clinical study showed a significant increase in depression in at least some of the patients enrolled in the trials. Also a recent article in the journal Multiple Sclerosis describes a patient whose previously subclinical case of multiple sclerosis became active when treatment with SR141716A was started.

Other compounds which have been identified as CB₁ and/or CB₂ cannabinoid receptor antagonists include the following: SR144528; O-2654; O-2050; NESS0327; AM281; AM251; LY320135; and AM630.

Naturally occurring CB₁ and CB₂ receptor antagonists which are produced by the cannabis plant are likely to have a less complex pharmacology than those of an inverse agonist which has been chemically synthesised to bind with the cannabinoid receptor. This is because the human body has been in contact with such substances for millennia and as such the body's pharmacological systems have developed in the presence of plant cannabinoids and if there were any untoward side effects these would be known already. However, until recently none of the cannabinoids produced by the cannabis plant have been found to possess inverse agonism properties of the cannabinoid receptor.

The applicant's co-pending patent application GB2434312 describes a THC extract which comprises a small amount of THCV, which is less than 2% (w/w). When the amount of THCV is lower than the amount of THC in the extract then it is impossible to determine what the effects of the THCV would be.

In the applicant's co-pending International patent application WO 2005/120478, the application describes that THCV could be used in place of THC. It has been subsequently found that this is not the case. THCV has been discovered to work as a CB₁ receptor antagonist, which is completely opposite from THC which acts as a CB₁ agonist.

The applicants have described in their co-pending application PCT/GB2005/004388 the cannabinoid receptor antagonist properties of the cannabinoid tetrahydrocannabidivarin (THCV). Here it is shown that the cannabinoid THCV acts as a neutral antagonist of the CB₁ and CB₂ cannabinoid receptors

More recently the applicants have described in their co-pending application PCT/GB2007/002008 the cannabinoid receptor antagonist properties of the cannabinoid cannabidiol (CBD). The cannabinoid CBD acts as an inverse agonist of the CB₁ and CB₂ cannabinoid receptors.

The applicants therefore believe that the combination of the cannabinoids tetrahydrocannabidivarin (THCV) and cannabidiol (CBD) will exhibit benefits as a pharmaceutical formulation as compared the use of each of the cannabinoids alone.

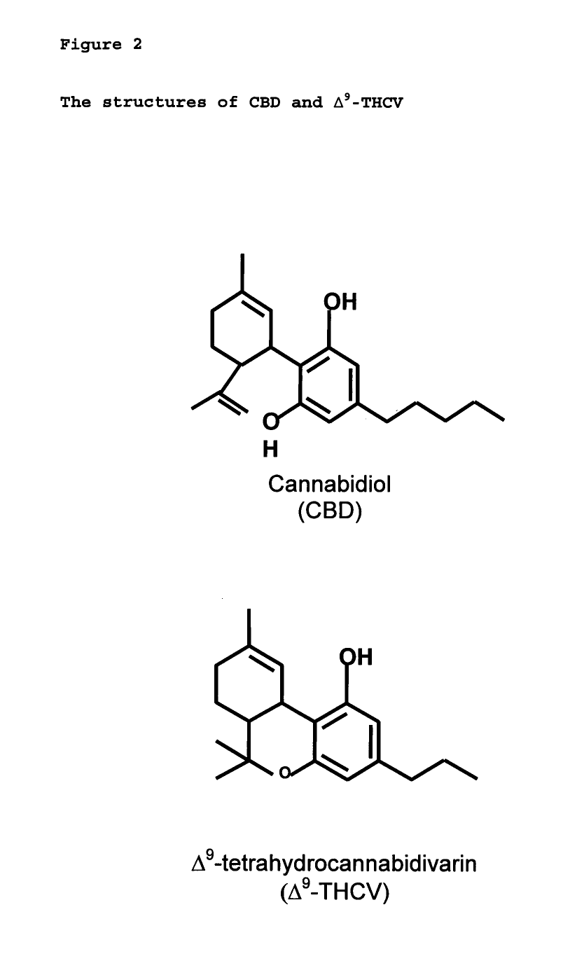

The cannabinoid THCV is a classical plant cannabinoid, which is structurally related to THC, in that instead of the 3-pentyl side chain of THC, the THCV molecule has a 3-propyl side chain. The cannabinoid CBD is again another classical plant cannabinoid, which is known to be non-psychoactive. CBD has previously been shown to be useful in the treatment of inflammation, nausea and anxiety. The structures of the two cannabinoids are shown in FIG. 2.

The two cannabinoids THCV and CBD can work together to provide a beneficial formulation, and this is of particular value. The diseases and conditions that the formulation with a combination of THCV and CBD will be useful in the treatment of are diseases and conditions that benefit from antagonism of the CB₁ and/or CB₂ cannabinoid receptors. It is thought that the combinations described herein provide a better treatment option due to the difference in the ways the two cannabinoids have an affect.

THCV is thought to act directly on the cannabinoid receptors and bind to cause a neutral antagonist effect. This means that the receptor itself is blocked to binding with an agonist such as an endocannabinoid; however the background tone of the receptor remains unaffected. When THCV is provided as a pharmaceutical formulation alone the unaffected background tone means that some of the diseases and conditions that antagonism is useful to treat may not be fully alleviated as the background tone may still cause an effect on the body.

Conversely, CBD is thought to act as an inverse agonist, which means that the background tone of the receptor is switched off. However, CBD is thought to bind at a site distinct from the cannabinoid receptors themselves and as such may allow an agonist to bind with the receptor.

A combination of the two cannabinoid receptor antagonists may therefore prove to be a very useful treatment option in diseases and conditions that benefit from antagonism of the CB₁ and/or CB₂ cannabinoid receptors.

### SUMMARY OF THE INVENTION

According to the first aspect of the present invention there is provided a pharmaceutical formulation comprising a ratioed mix of: (i) one or more compounds that acts as an inverse agonist of the CB₁ and/or CB₂ receptor; and (ii) one or more compounds that acts as a neutral antagonist of the CB₁ and/or CB₂ receptor.

The above ratioed mix will include the alternatives as follows:

A ratioed mix of: (i) one or more compounds that acts as an inverse agonist of the CB₁ receptor; and (ii) one or more compounds that acts as a neutral antagonist of the CB₁ receptor;

A ratioed mix of: (i) one or more compounds that acts as an inverse agonist of the CB₁ receptor; and (ii) one or more compounds that acts as a neutral antagonist of the CB₂ receptor;

A ratioed mix of: (i) one or more compounds that acts as an inverse agonist of the CB₂ receptor; and (ii) one or more compounds that acts as a neutral antagonist of the CB₁ receptor;

A ratioed mix of: (i) one or more compounds that acts as an inverse agonist of the CB₂ receptor; and (ii) one or more compounds that acts as a neutral antagonist of the CB₂ receptor;

A ratioed mix of: (i) one or more compounds that acts as an inverse agonist of both the CB₁ and the CB₂ receptors; and (ii) one or more compounds that acts as a neutral antagonist of both the CB₁ and the CB₂ receptors;

A ratioed mix of: (i) one or more compounds that acts as an inverse agonist of both the CB₁ and the CB₂ receptors; and (ii) one or more compounds that acts as a neutral antagonist of the CB₁ receptor;

A ratioed mix of: (i) one or more compounds that acts as an inverse agonist of both the CB₁ and the CB₂ receptors; and (ii) one or more compounds that acts as a neutral antagonist of the CB₂ receptor;

A ratioed mix of: (i) one or more compounds that acts as an inverse agonist of the CB₁ receptor; and (ii) one or more compounds that acts as a neutral antagonist of both the CB₁ and the CB₂ receptors; and

A ratioed mix of: (i) one or more compounds that acts as an inverse agonist of the CB₂ receptor; and (ii) one or more compounds that acts as a neutral antagonist of both the CB₁ and the CB₂ receptors.

Preferably the pharmaceutical formulation comprises a cannabinoid which acts as inverse agonist of the CB₁ and/or CB₂ receptor.

More preferably the cannabinoid which is an inverse agonist of the CB₁ and/or CB₂ receptor is cannabidiol (CBD).

Preferably the pharmaceutical formulation comprises a cannabinoid which acts as a neutral antagonist of the CB₁ and/or CB₂ receptor.

More preferably the cannabinoid which is a neutral antagonist of the CB₁ and/or CB₂ receptor is tetrahydrocannabidivarin (THCV).

More preferably still, the ratioed mix of (i) and (ii) is a ratioed mix of THCV and CBD.

Such a pharmaceutical formulation may used in the manufacture of a medicament for the treatment of diseases such as obesity, schizophrenia, epilepsy or cognitive disorders such as Alzheimer's, bone disorders, bulimia, obesity associated with type II diabetes (non-insulin dependant diabetes) and in the treatment of drug, alcohol or nicotine abuse or dependency. These diseases may be caused by agonism of the CB₁ receptor and therefore can be treated with different ratioed mixtures of the inverse agonist and neutral antagonist of the CB₁ receptor.

Inflammatory diseases may be caused by agonism of the CB₂ receptor can also be treated with different ratioed mixtures of the inverse agonist and neutral antagonist of the CB₂ receptor.

Such formulations may be of particular value in the treatment of diseases with multiple symptoms as the combined mixture of inverse agonist of the CB₁ and/or CB₂ receptor and neutral antagonist of the CB₁ and/or CB₂ receptor will provide a dual benefit.

The rationale behind producing a formulation which has the properties of both neutral antagonism and inverse agonism of the CB₁ or CB₂ receptors is to enable diseases which would normally be treated by either a neutral antagonist or an inverse agonist to have an enhanced treatment option.

For example, as has already been described by the applicants in their co-pending application (PCT/GB05/004388), THCV is useful in producing beneficial weight loss in obese mammals. This appears to be due to an increase in the energy expenditure and food conversion efficiency. It is thought that THCV achieves such properties by antagonism of the CB₁ receptor. Unfortunately there are associated problems with the treatment of diseases such as obesity with THCV due to the ongoing background tone in the cells of mammals suffering from obesity. A treatment option that combines THCV with an inverse CB₁ agonist which is able to switch off the background tone of the cells provides a valuable solution.

The combination of a neutral antagonist and an inverse agonist enables the treatment of obese animals. The combination results in a lowered blood triglyceride level and in consequence an increase in HDL-cholesterol (which is often referred to as ‘good cholesterol’).

The combination of a neutral antagonist and an inverse agonist also enables the treatment of diabetic animals. The combination results in a reduction in plasma insulin levels and improved glucose tolerance.

References to THCV and CBD, THCV- and CBD-type compounds or derivatives thereof, particularly with regard to therapeutic use, will be understood to also encompass pharmaceutically acceptable salts of such compounds. The term “pharmaceutically acceptable salts” refers to salts or esters prepared from pharmaceutically acceptable non-toxic bases or acids, including inorganic bases or acids and organic bases or acids, as would be well known to persons skilled in the art. Many suitable inorganic and organic bases are known in the art.

The scope of the invention also extends to derivatives of THCV or CBD that retain the desired activity of neutral antagonism or inverse agonism of the CB₁ and/or CB₂ receptor. Derivatives that retain substantially the same activity as the starting material, or more preferably exhibit improved activity, may be produced according to standard principles of medicinal chemistry, which are well known in the art. Such derivatives may exhibit a lesser degree of activity than the starting material, so long as they retain sufficient activity to be therapeutically effective. Derivatives may exhibit improvements in other properties that are desirable in pharmaceutically active agents such as, for example, improved solubility, reduced toxicity, enhanced uptake.

Preferably the THCV and CBD are in the form of a cannabinoid-containing plant extract from at least one cannabis plant.

More preferably the cannabinoid-containing plant extract from at least one cannabis plant is a botanical drug substance.

In one embodiment the cannabinoid-containing plant extract from at least one cannabis plant is produced by extraction with supercritical or subcritical CO₂.

Alternatively the cannabinoid-containing plant extract from at least one cannabis plant is produced by contacting plant material with a heated gas at a temperature which is greater than 100° C., sufficient to volatilise one or more of the cannabinoids in the plant material to form a vapour, and condensing the vapour to form an extract.

Preferably the cannabinoid-containing plant extract from at least one cannabis plant comprises all the naturally occurring cannabinoids in the plant.

Alternatively the THCV and/or CBD are in a substantially pure or isolated form.

A “substantially pure” preparation of cannabinoid is defined as a preparation having a chromatographic purity (of the desired cannabinoid) of greater than 90%, more preferably greater than 95%, more preferably greater than 96%, more preferably greater than 97%, more preferably greater than 98%, more preferably greater than 99% and most preferably greater than 99.5%, as determined by area normalisation of an HPLC profile.

Preferably the substantially pure cannabinoid used in the invention is substantially free of any other naturally occurring or synthetic cannabinoids, including cannabinoids which occur naturally in cannabis plants. In this context “substantially free” can be taken to mean that no cannabinoids other than the target cannabinoid are detectable by HPLC.

Particularly in the case of THCV, it is known that the cannabinoid THCV is produced together with THC in the cannabis plant. The psychoactive side effects of THC are not wanted especially when producing a pharmaceutical formulation and as such the plant extracts used in the formulations of the invention can be selectively treated to remove other cannabinoids such as THC.

In another aspect of the present invention the cannabinoids are in a synthetic form.

Preferably the pharmaceutical formulation further comprises one or more pharmaceutically acceptable carriers, excipients or diluents.

The invention also encompasses pharmaceutical formulations, formulated into pharmaceutical dosage forms, together with suitable pharmaceutically acceptable carriers, such as diluents, fillers, salts, buffers, stabilizers, solubilizers, etc. The dosage form may contain other pharmaceutically acceptable excipients for modifying conditions such as pH, osmolarity, taste, viscosity, sterility, lipophilicity, solubility etc. The choice of diluents, carriers or excipients will depend on the desired dosage form, which may in turn be dependent on the intended route of administration to a patient.

Suitable dosage forms include, but are not limited to, solid dosage forms, for example tablets, capsules, powders, dispersible granules, cachets and suppositories, including sustained release and delayed release formulations. Powders and tablets will generally comprise from about 5% to about 70% active ingredient. Suitable solid carriers and excipients are generally known in the art and include, e.g. magnesium carbonate, magnesium stearate, talc, sugar, lactose, etc. Tablets, powders, cachets and capsules are all suitable dosage forms for oral administration.

Liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Liquid dosage forms also include solutions or sprays for intranasal, buccal or sublingual administration. Aerosol preparations suitable for inhalation may include solutions and solids in powder form, which may be combined with a pharmaceutically acceptable carrier, such as an inert compressed gas.

Also encompassed are dosage forms for transdermal administration, including creams, lotions, aerosols and/or emulsions. These dosage forms may be included in transdermal patches of the matrix or reservoir type, which are generally known in the art.

Pharmaceutical preparations may be conveniently prepared in unit dosage form, according to standard procedures of pharmaceutical formulation. The quantity of active compound per unit dose may be varied according to the nature of the active compound and the intended dosage regime. Generally this will be within the range of from 0.1 mg to 1000 mg.

It may be preferable depending on the disease or condition which is to be treated to have a high dose of the inverse agonist the CB₁ and/or CB₂ receptor and a low dose of the neutral antagonist of the CB₁ and/or CB₂ receptor, or vice versa. For example a high dose of CBD of 1000 mg may be combined with a low dose of THCV of 10 mg. Alternatively the dose of each inverse agonist or neutral antagonist may be approximately the same.

Preferably the ratio of (i):(ii) in the pharmaceutical formulation is from 99:1 to 1:99.

Preferably the ratio of THCV and CBD in the pharmaceutical formulation are in a ratio of from 99:1 and 1:99 THCV:CBD (w/w).

More preferably the ratio of THCV:CBD is from 85:15 to 15:85 THCV:CBD (w/w).

More preferably the ratio of THCV:CBD is from 75:25 to 25:75 THCV:CBD (w/w).

More preferably the ratio of THCV:CBD is from 65:35 to 35:65 THCV:CBD (w/w).

More preferably the ratio of THCV:CBD is from 55:45 to 45:55 THCV:CBD (w/w).

More preferably the ratio of THCV:CBD is approximately 50:50 THCV:CBD (w/w).

Certain aspects of this invention are further described, by way of example only, with reference to the accompanying drawings in which:

FIG. 1 shows the agonism and antagonism of constitutively active receptors; and

FIG. 2 shows the 2-dimensional structure of the cannabinoid tetrahydrocannabidivarin (THCV) and cannabidiol (CBD).

### SPECIFIC DESCRIPTION

The examples described below relate to the preparation of a dosage form containing a mixture of extracts of cannabis. The extracts are referred to as cannabis-based medicinal extracts (CBME) for ease of reference.

An extract from a chemovar of cannabis producing cannabidiol (CBD) as a main cannabinoid and an extract from a chemovar producing tetrahydrocannabidivarin (THCV) as a main cannabinoid have been used in many of the examples below. These cannabinoids were used to produce formulations as the binding properties of these cannabinoids have been explored; the data from these experiments is detailed in Example 1.

The remainder of the examples describe different types of pharmaceutical formulations that may be useful for administration of a neutral antagonist of the CB₁ and/or CB₂ receptor combined with an inverse agonist of the CB₁ and/or CB₂ receptor.

The formulas described in these examples can be varied to accommodate CBME with a greater or lesser amount of cannabinoid in order to achieve the desired ratio of THCV to CBD or other cannabinoids or active agents. Different ratios of neutral antagonists of the CB₁ and/or CB₂ receptor and inverse agonists of the CB₁ and/or CB₂ receptor will be useful in the treatment of specific therapeutic conditions.

### Example 1

Experiments were performed with membranes prepared from healthy brain tissue, which is densely populated with CB₁ but not CB₂ receptors. Further experiments were undertaken with Chinese hamster ovary (CHO) cells transfected with hCB₂ receptors. These membranes were used to investigate the ability of the test compound to displace [³H]CP55940 CB₂ binding sites

These experiments were used to determine whether the test compounds behaved as a CB₁ and/or a CB₂ receptor agonist or antagonist. For these experiments the test compounds used were THCV (cannabinoid-containing plant extract) and CBD (cannabinoid-containing plant extract), both singly and as a mixture.

Methods:

Radioligand Displacement Assay

The assays were carried out with [³H]CP55940, 1 mg ml⁻¹ bovine serum albumin (BSA) and 50 mM Tris buffer, total assay volume 500 μl.

Binding was initiated by the addition of either the brain membranes (33 μg protein per tube) or the transfected hCB₂ cells (25 μg protein per tube).

All assays were performed at 37° C. for 60 min before termination by addition of ice-cold wash buffer (50 mM Tris buffer, 1 mg ml⁻¹ bovine serum albumin, pH 7.4) and vacuum filtration using a 24-well sampling manifold and GF/B filters that had been soaked in wash buffer at 4° C. for at least 24 h.

Each reaction tube was washed six times with a 1.2 ml aliquot of wash buffer. The filters were oven-dried for 60 min and then placed in 5 ml of scintillation fluid. Radioactivity was quantified by liquid scintillation spectrometry.

Specific binding was defined as the difference between the binding that occurred in the presence and absence of 1 μM unlabelled CP55940. The THCV and CBD were stored as a stock solution of 10 mM in DMSO, the vehicle concentration in all assay tubes being 0.1% DMSO.

The binding parameters for [³H]CP55940, were 2336 fmol mg⁻¹ protein (Bₘₐₓ) and 2.31 nM (K_{d}) in mouse brain membranes, and 72570 fmol/mg protein (Bₘₐₓ) and 1.043 nM (K_{d}) in hCB₂ transfected cells.

[³⁵S]GTPγS Binding Assay

The assays were carried out with GTPγS binding buffer (50 mM Tris-HCl; 50 mM Tris-Base; 5 mM MgCl₂; 1 mM EDTA; 100 mM NaCl; 1 mM DTT; 0.1% BSA) in the presence of [³⁵S]GTPγS and GDP, in a final volume of 500 μl. Binding was initiated by the addition of [³⁵S]GTPγS to the tubes. Nonspecific binding was measured in the presence of 30 μM GTPγS.

The drugs were incubated in the assay for 60 min at 30° C. The reaction was terminated by a rapid vacuum filtration method using Tris buffer (50 mM Tris-HCl; 50 mM Tris-Base; 0.1% BSA), and the radioactivity was quantified by liquid scintillation spectrometry.

The concentrations of [³⁵S]GTPγS and GDP present in the assay varied depending on whether the assay was conducted with mouse brain or transfected cell membranes. When the assay was conducted with mouse brain membranes, 0.1 nM [³⁵S]GTPγS and 30 μM GDP were present, whereas the corresponding concentrations present when the assay was conducted with transfected cell membranes were 1 nM and 320 μM respectively.

Additionally, mouse brain membranes were preincubated for 30 minutes at 30° C. with 0.5 U ml⁻¹ adenosine deaminase to remove endogenous adenosine. Agonists and antagonists were stored as a stock solution of 1 or 10 mM in DMSO, the vehicle concentration in all assay tubes being 0.11% DMSO.

Analysis of Data

Values are expressed as means and variability as s.e.mean or as 95% confidence limits. The concentration of THCV that produced a 50% displacement of radioligand from specific binding.

Net agonist-stimulated [³⁵S]GTPγS binding values were calculated by subtracting basal binding values (obtained in the absence of agonist) from agonist-stimulated values (obtained in the presence of agonist) as detailed elsewhere (Ross et al., 1999a).

Inhibition of the electrically-evoked twitch response of the vas deferens has been expressed in percentage terms and this has been calculated by comparing the amplitude of the twitch response after each addition of a twitch inhibitor with its amplitude immediately before the first addition of the inhibitor. Contractile responses to phenylephrine and β,γ-methylene-ATP have been expressed as increases in tension (g).

Values for EC₅₀, for maximal effect (Eₘₐₓ) and for the s.e.mean or 95% confidence limits of these values have been calculated by nonlinear regression analysis using the equation for a sigmoid concentration-response curve (GraphPad Prism).

The apparent dissociation constant (K_{B}) values for antagonism of agonists by THCV in the vas deferens or [³⁵S]GTPγS binding assay have been calculated by Schild analysis from the concentration ratio, defined as the concentration of an agonist that elicits a response of a particular size in the presence of a competitive reversible antagonist at a concentration, B, divided by the concentration of the same agonist that produces an identical response in the absence of the antagonist.

The methods used to determine concentration ratio and apparent K_{B} values and to establish whether log concentration-response plots deviated significantly from parallelism are detailed elsewhere (Pertwee et al., 2002). Mean values have been compared using Student's two-tailed t-test for unpaired data or one-way analysis of variance (ANOVA) followed by Dunnett's test (GraphPad Prism). A P-value <0.05 was considered to be significant.

Results:

THCV

THCV displaced [³H]CP55940 from specific binding sites in mouse brain and CHO-hCB₂ cell membranes in a manner that fitted significantly better to a one-site than a two-site competition curve (P<0.05; GraphPad Prism 4). Its mean Kᵢ values were 75.4 nM and 62.8 nM respectively.

THCV also displaced [³H]R-(+)-WIN55212 and [³H]SR141716A from specific binding sites in mouse brain membranes, its mean EC₅₀ values with 95% confidence limits shown in brackets being 61.3 nM (48.6 and 77.3 nM; n=4 to 7) and 86.8 nM (63.8 and 188.1 nM; n=4 to 6) respectively. The corresponding EC₅₀ value of THCV for displacement of [³H]CP55940 is 98.2 nM (69.6 and 138.6 nM; n=4 to 8).

The ability of CP55940 to enhance [³⁵S]GTPγS binding to mouse brain and CHO-hCB₂ membranes was attenuated by THCV, which at 1 μM produced significant dextral shifts in the log concentration response curves of this cannabinoid receptor agonist that did not deviate significantly from parallelism.

The mean apparent K_{B} values for this antagonism are shown in Table 1, as are mean apparent K_{B} values of SR141716A for antagonism of CP55940 in mouse brain membranes and of SR144528 for antagonism of CP55940 in the CHO-hCB₂ cell membranes. At 1 μM, THCV also produced a significant parallel dextral shift in the log concentration response curve of R-(+)-WIN55212 for enhancement of GTPγS binding to mouse brain membranes.

TABLE 1

| | | | Mean | 95% | |
|---|---|---|---|---|---|
| | | | apparent | confidence | |
| | | Membrane | K_{B} | limits | |
| Antagonist | Agonist | preparation | (nM) | (nM) | n |
| THCV | CP55940 | Brain | 93.1 | 66.5, | 6 |
| (1000 nM) | | | | 130.6 | |
| THCV | R-(+)- | Brain | 85.4 | 29.3, | 5 |
| (1000 nM) | WIN55212 | | | 270.5 | |
| SR141716A | CP55940 | Brain | 0.09 | 0.021, | 4 |
| (10 nM) | | | | 0.41 | |
| THCV | CP55940 | CHO—hCB₂ | 10.1 | 5.0, | 6 |
| (1000 nM) | | | | 20.5 | |
| SR144528 | CP55940 | CHO—hCB₂ | 0.49 | 0.26, | 6 |
| (100 nM) | | | | 0.85 | |

CBD

Table 2 describes the data produced by CBD and the known CB₁ receptor inverse agonist SR141716A at the CB₁ receptor.

The table describes the K_{B}-values for the CP55940 induced activation of [³⁵S]GTPγS binding to the cell membrane in the presence of the known CB₁ receptor inverse agonist and CBD.

The Kᵢ-value for the displacement of the [³H]CP55940 from the membranes is also shown.

TABLE 2

| | K_{B}-value for | Kᵢ-value for |
|---|---|---|
| Test Article | Binding | Displacement |
| SR141716A | 0.09 nM | 2.2 nM |
| (10 nM) | | |
| CBD | 78.8 nM | 4.9 μM |
| (1 μM) | | |

Both SR141617A and CBD were able to produce a rightward shift in the log-concentration response curve of the established CB₁/CB₂ receptor agonist CP55940 in the mouse brain membranes when the measured response was stimulation of [³⁵S]GTPγS binding. These data show that both compounds were able to inhibit the response caused by the activation of the CB₁ receptor by CP55940.

The K_{B}-value of SR141716A was 0.09 nM which is only slightly less than its CB₁ Kᵢ-value of 2.2 nM for the displacement of [³H]CP55940 from the mouse brain membranes. This infers that this compound is able to produce an inverse response in the cell at a similar concentration to that at which it competes and binds to the receptor.

However the K_{B}-value of CBD was 78.8 nM this was well below its CB₁ Kᵢ-value of 4.9 μM for the displacement of [³H]CP55940 from the mouse brain membranes. These data show that CBD is able to act as an inverse agonist at the CB₁ receptor. They also show that CBD is able to act as an inverse agonist at concentrations much below that at which it will compete with the agonist for the binding site.

This property may be of significant value as it infers that CBD will form a less strong interaction with the cannabinoid receptor in vivo and as such is likely to produce fewer side effects in use than the compound SR141716A.

Further experiments were undertaken at different concentrations of the test compounds. At concentrations of 1 and 10 μM CBD produced a significant inhibition of [³⁵S]GTPγS binding to the mouse brain membrane. The inhibitory effect of CBD at 1 μM was similar to that of SR141716A at 1 μM, whereas the inhibitory effect of CBD at 10 μM greatly exceeded that of SR141716A at the same concentration. At the higher concentration CBD is a more potent inverse agonist of the CB₁ receptor than SR141716A.

Table 3 describes the data produced by CBD and the known CB₂ receptor inverse agonist SR144528 at the CB₂ receptor.

The table describes the K_{B}-values for the CP55940 induced activation of [³⁵S]GTPγS binding to the cell membrane in the presence of the known CB₁ receptor inverse agonist and CBD. The Kᵢ-value for displacement of the [³H]CP55940 from the membranes is also shown.

TABLE 3

| | K_{B}-value for | Kᵢ-value for |
|---|---|---|
| Test Article | Binding | Displacement |
| SR144528 | 0.49 nM | 7.5 nM |
| (100 nM) | | |
| CBD | 65.1 nM | 4.2 μM |
| (1 μM) | | |

Both SR144528 and CBD were able to produce a downward and rightward shift in the log-concentration response curve of the established CB₁/CB₂ receptor agonist CP55940 in the CHO cell membranes when the measured response was stimulation of [³⁵S]GTPγS binding. These data show that both compounds were able to inhibit the response caused by the activation of the CB₂ receptor by CP55940.

The K_{B}-value of SR144528 was 0.49 nM which was 15 times less than its CB₁ Kᵢ-value of 7.5 nM for the displacement of [³H]CP55940 from the CHO cell membranes.

The K_{B}-value of CBD was 65.1 nM which was 65 times less than its CB₁ Kᵢ-value of 4.2 μM for the displacement of [³H]CP55940 from the CHO cell membranes.

Conclusions:

Δ⁹-tetrahydrocannabivarin (THCV) displaced [³H]CP55940 from specific binding sites on brain and CHO-hCB₂ cell membranes Kᵢ=75.4 and 62.8 nM respectively), indicating that THCV is both a CB₁ and CB₂ receptor antagonist.

THCV (1 μM) also antagonized CP55940-induced enhancement of [³⁵S]GTPγS binding to these membranes (apparent K_{B}=93.1 and 10.1 nM respectively), indicating that it is a reasonably potent competitive antagonist. The K_{B} values indicate that THCV is more potent as a CB₂ than a CB₁ receptor antagonist.

THCV produced its antagonism of cannabinoids at concentrations that by themselves did not affect the amplitude of the electrically-evoked contractions, or the ability of [³⁵S]GTPγS to bind to mouse brain membranes or CHO-hCB2 cell membranes, suggesting that THCV is a neutral cannabinoid receptor antagonist.

CBD is able to act as an inverse agonist at the CB₁ and CB₂ receptors. CBD acts as inverse agonist at concentrations below that at which it competes with the agonist for the binding site. However CBD was shown to compete at a far lower concentration than SR144528.

In summary the data produced in this example indicates that CBD is an inverse agonist at both the CB₁ and CB₂ receptors. It is also shown that CBD will only displace agonists from their cannabinoid receptor binding sites at far higher concentrations than that at which it is able to produce the inverse agonism in the cell.

### Example 2

A mixture is prepared by melting together the following ingredients:

| | | |
|---|---|---|
| Glycerol mono-oleate | 10 | parts |
| Soy lecithin | 5 | parts |
| CBME - to give CBD | 1 | part |
| CBME - to give THCV | 2 | parts |
| Alpha-tocopherol | 0.1 | part |
| Ascorbyl palmitate BP | 0.1 | part |
| Glycogelatin to produce | 100 | parts |

The components are mixed together over a gentle heat and poured into moulds whilst hot. The product in moulds is formed into a rigid gel and sealed in an inert atmosphere. The relatively large size of this dosage form (1-2 g) allows a large amount of active ingredient to be incorporated into the dosage form. Each dose unit may be administered by allowing to dissolve in the mouth, sublingually, buccally or swallowed whole or in smaller units.

### Example 3

A smaller unit dosage form may be prepared using the following example, whereby a smaller amount of active can be incorporated. The following example is particularly suitable for an oral dosage form such as a tablet.

| | | |
|---|---|---|
| Glycerol monosterate (self emulsifying grade) | 5 | parts |
| Polysorbate 80 | 0.5 | parts |
| Lactose (direct compression grade) | 79.3 | parts |
| Soluble starch | 10 | parts |
| CBME - to give CBD | 2.5 | parts |
| CBME - to give THCV | 2.5 | parts |
| Ascorbyl palmitate | 0.1 | part |
| Alpha-tocopherol | 0.1 | part |
| Ethanol (dehydrated) BP | 10 | parts |

The glycerol monosterate, polysorbate, alpha-tocopherol and CBMEs are dispersed and dissolved in the ethanol. This solution is then sprayed onto the dry poweder ingredients which have been thoroughly mixed. The ethanol is allowed to evaporate and the granules are dusted with 1% talc and compressed to the target tablet weight of 101 mg in a conventional tablet press. Biconvex punches with a diameter of 7-9 mm are used to produce tablets with a high surface to weight ratio. These are able to absorb water when placed under the sublingual or buccal mucosae and disperse in a period of 30 seconds to 5 minutes.

Alternatively the tablets may be swallowed whole as an oral dosage form.

### Example 4

The generation of an emulsion from a self-emulsifying formulation is not limited to solid dosage forms. In the following example three liquid formulations suitable for sublingual application are exemplified. A solution is produced by melting together, at a temperature not exceeding 50° C., the following ingredients:

| | A | B | C | D | E |
|---|---|---|---|---|---|
| Glycerol mono-oleate | 2 | 2 | 2 | 2 | 2 |
| (self-emulsifying) | | | | | |
| Medium chain triglyceride | 5 | — | — | — | — |
| Cremophor RH40 | 30 | 26.5 | — | — | — |
| CBME - to give CBD | 5 | 1 | 9 | 7.5 | 2.5 |
| CBME - to give THCV | 5 | 9 | 1 | 2.5 | 7.5 |
| Alpha-tocopherol | 0.1 | 0.1 | 0.1 | 0.1 | 0.1 |
| Ascorbyl palmitate | 0.1 | 0.1 | 0.1 | 0.1 | 0.1 |
| Propylene glycol | — | — | 44 | — | — |
| Ethanol (to give) | 100 | 100 | 100 | 100 | 100 |

The products formed by mixing these ingredients are dispersed in 10 ml quantities into a glass vial ad closed with a pump action break-up button. Each actuation of the pump delivers a fine spray which can be directed to an area of the buccal or sublingual mucosae or can be simply sprayed into the mouth and swallowed.

Solutions based on ethanol alone are generally not suitable to be used as a mouth spray. The addition of a self-emulsifying agent allows this problem to be overcome.

### Example 5

The solid dosage form may be a soft gelatine capsule which can be crushed to release the medicament to give an emulsion or swallowed orally. The soft gelatine capsule described below provides an emulsified form of medicament which can be absorbed from any part of the GI tract.

| | | |
|---|---|---|
| Glycerol monosterate (self emulsifying grade) | 5 | parts |
| Polysorbate 80 | 1 | part |
| Beeswax | 5 | parts |
| CBME - to give CBD | 10 | parts |
| CBME - to give THCV | 10 | parts |
| Ascorbyl palmitate | 0.1 | part |
| Alpha-tocopherol | 0.1 | part |
| Hemp oil (to produce) | 100 | parts |

### Example 6

A dosage form as described above which uses vegetable rather than animal gelling agents may be made as follows:

| | | |
|---|---|---|
| Sorbitol | 35 | parts |
| Gum acacia | 20 | parts |
| Glycerol mono-oleate | 10 | parts |
| Egg lecithin | 10 | parts |
| CBME - to give CBD | 2.5 | parts |
| CBME - to give THCV | 2.5 | parts |
| Ascorbyl palmitate | 0.1 | part |
| Alpha-tocopherol | 0.1 | part |
| Ethanol (dehydrated) BP | 10 | parts |
| Vanillin | 0.1 | parts |
| BHT | 0.01 | parts |
| Glycerol | 5 | parts |
| Water (to give) | 100 | parts |

The fat soluble ingredients are melted together at a temperature of 70° C. Sorbitol is mixed with the acacia gum, dispersed in glycerol, and added to the other solid ingredients. Water is added and the mass heated on a boiling water bath until evenly dispersed. While still at a temperature of 60° C. the mass can be distributed into moulds.

### Example 7

A product providing a fast release of one constituent and a slower release of another constituent can be produced by making a combination dose unit. Using the formulation described in example 5 a quantity of heated mass is filled into a mould or cast into a film, and allowed to set. A layer of material as described in example 2 is then cast onto the surface of the gel. Variations of the proportions and active content in the two layers provides opportunities for the treatment of different diseases and conditions where the administration of either a neutral antagonist of the CB₁ and/or CB₂ receptor is useful either before or after the administration of an inverse agonist of the CB₁ and/or CB₂ receptor.

### Example 8

The example described below details the features of formulations intended for spray application to the buccal mucosae.

A solution is produced by dissolving the following ingredients at a temperature not exceeding 50° C.

| | A | B | C | D | E |
|---|---|---|---|---|---|
| Glycerol monostearate | 2 | — | 2 | — | 2 |
| (self-emulsifying) | | | | | |
| Glycerol mono-oleate | — | 2 | — | 2 | 2 |
| Cremophor RH40 | 20 | 30 | 30 | 20 | 30 |
| CBME - to give CBD | 5 | 2.5 | 5 | 1.5 | 3.5 |
| CBME - to give THCV | 5 | 5 | 2.5 | 3.5 | 1.5 |
| Alpha-tocopherol | 0.1 | 0.1 | 0.1 | 0.1 | 0.1 |
| Ascorbyl palmitate | 0.1 | 0.1 | 0.1 | 0.1 | 0.1 |
| Ethanol (to give) | 100 | 100 | 100 | 100 | 100 |

The product formed by mixing together these ingredients is dispensed into glass vials and closed with a pump action or aerosol spray.

### Example 9

The example described below details the features of formulations which can be dispensed from a pump action spray device. The product can be dispensed to produce a ribbon of gel which can either be swallowed or can be applied to the buccal or other mucosae.

| | | |
|---|---|---|
| Carboxymethylcellulose sodium | 2 | parts |
| Glycerol monosterate (self emulsifying grade) | 10 | parts |
| Glycerol | 10 | parts |
| CBME - to give CBD | 10 | parts |
| CBME - to give THCV | 10 | parts |
| Ascorbic acid | 0.1 | part |
| Alpha-tocopherol | 0.1 | part |
| Water (to produce) | 100 | parts |

The non-aqueous ingredients are melted together at a temperature of not more than 50° C. until evenly suspended. Water is then added to form a creamy gel. The product is dispensed into containers whilst still warm and sealed with a pump dispenser head.

### Example 10

The example described below details the features of formulations produced with less than 5% water. The presence of water can sometimes cause precipitation of the active ingredients. The product can be dispensed from a pump action spray device. The product can be dispensed to produce a spray which can either be swallowed or can be applied to the buccal or other mucosae.

| | | |
|---|---|---|
| Propylene glycol | 50 | parts |
| CBME - to give CBD | 2.5 | parts |
| CBME - to give THCV | 2.5 | parts |
| Peppermint oil | 0.005 | part |
| Ethanol (to produce) | 100 | parts |

1. A pharmaceutical formulation comprising a ratioed mix of: (i) one or more compounds that acts as an inverse agonist of the CB₁ and/or CB₂ receptor; and (ii) one or more compounds that acts as a neutral antagonist of the CB₁ and/or CB₂ receptor.

2. A pharmaceutical formulation as claimed in claim 1, comprising a ratioed mix of: (i) one or more compounds that acts as an inverse agonist of the CB₁ receptor; and (ii) one or more compounds that acts as a neutral antagonist of the CB₁ receptor.

3. A pharmaceutical formulation as claimed in claim 1 comprising a ratioed mix of: (i) one or more compounds that acts as an inverse agonist of the CB₁ receptor; and (ii) one or more compounds that acts as a neutral antagonist of the CB₂ receptor.

4. A pharmaceutical formulation as claimed in claim 1, comprising a ratioed mix of: (i) one or more compounds that acts as an inverse agonist of the CB₂ receptor; and (ii) one or more compounds that acts as a neutral antagonist of the CB₁ receptor.

5. A pharmaceutical formulation as claimed in claim 1, comprising a ratioed mix of: (i) one or more compounds that acts as an inverse agonist of the CB₂ receptor; and (ii) one or more compounds that acts as a neutral antagonist of the CB₂ receptor.

6. A pharmaceutical formulation as claimed in claim 1, comprising a ratioed mix of: (i) one or more compounds that acts as an inverse agonist of both the CB₁ and the CB₂ receptors; and (ii) one or more compounds that acts as a neutral antagonist of both the CB₁ and the CB₂ receptors.

7. A pharmaceutical formulation as claimed in claim 1, comprising a ratioed mix of: (i) one or more compounds that acts as an inverse agonist of both the CBi and the CB₂ receptors; and (ii) one or more compounds that acts as a neutral antagonist of the CB₁ receptor.

8. A pharmaceutical formulation as claimed in claim 1, comprising a ratioed mix of: (i) one or more compounds that acts as an inverse agonist of both the CB₁ and the CB₂ receptors; and (ii) one or more compounds that acts as a neutral antagonist of the CB₂ receptor.

9. A pharmaceutical formulation as claimed in claim 1, comprising a ratioed mix of: (i) one or more compounds that acts as an inverse agonist of the CB₁ receptor; and (ii) one or more compounds that acts as a neutral antagonist of both the CB₁ and the CB₂ receptors.

10. A pharmaceutical formulation as claimed in claim 1, comprising a ratioed mix of: (i) one or more compounds that acts as an inverse agonist of the CB₂ receptor; and (ii) one or more compounds that acts as a neutral antagonist of both the CB₁ and the CB₂ receptors.

11. A pharmaceutical formulation as claimed in claim 1, wherein the inverse agonist of the CB₁ and/or CB₂ receptor is a cannabinoid.

12. A pharmaceutical formulation as claimed in claim 11, wherein the inverse agonist of the CB₁ and/or CB₂ receptor is cannabidiol (CBD).

13. A pharmaceutical formulation as claimed in claim 1, wherein the neutral antagonist of the CB₁ and/or CB₂ receptor is a cannabinoid.

14. A pharmaceutical formulation as claimed in claim 13, wherein the neutral antagonist of the CB₁ and/or CB₂ receptor is tetrahydrocannabidivarin (THCV).

15. A pharmaceutical formulation as claimed in claim 1, wherein the ratioed mix of (i) and (ii) is a ratioed mix of THCV and CBD.

16. A pharmaceutical formulation as claimed in claim 15, wherein the THCV and CBD are in the form of one or more cannabinoid-containing plant extract from at least one cannabis plant.

17. A pharmaceutical formulation as claimed in claim 16, wherein the cannabinoid-containing plant extract from at least one cannabis plant is a botanical drug substance.

18. A pharmaceutical formulation as claimed in claim 16, wherein the cannabinoid-containing plant extract from at least one cannabis plant comprises all the naturally occurring cannabinoids in the plant.

19. A pharmaceutical formulation as claimed in claim 15, wherein the THCV and/or CBD are in a substantially pure or isolated form.

20. A pharmaceutical formulation as claimed in claim 15, wherein the THCV and/or CBD are in a synthetic form.

21. A pharmaceutical formulation as claimed in claim 1, wherein the formulation further comprises one or more pharmaceutically acceptable carriers, excipients or diluents.

22. A pharmaceutical formulation as claimed in claim 1, wherein the formulation is administered using one or more of the following: tablets, capsules, powders, dispersible granules, cachets and suppositories, sustained release and delayed release formulations, liquid dosage forms, solutions, suspensions and emulsions, injectable formulations, solutions or sprays for intranasal, buccal or sublingual administration, aerosol preparations suitable for inhalation, transdermal formulations, creams, lotions, aerosols and/or emulsions and transdermal patches.

23. A pharmaceutical formulation as claimed in claim 1, wherein the quantity of active compound per unit dose is within the range of from 0.1 mg to 1000 mg.

24. A pharmaceutical-formulation as claimed in claim 1, wherein the ratio of (i):(ii) is from 99:1 to 1:99 (w/w).

25. A pharmaceutical formulation as claimed in claim 15, wherein the THCV and CBD in the pharmaceutical formulation are in a ratio of from 99:1 to 1:99 THCV:CBD (w/w).

26. A pharmaceutical formulation as claimed in claim 15, wherein the THCV and CBD in the pharmaceutical formulation are in a ratio of from 85:15 to 15:85 THCV:CBD (w/w).

27. A pharmaceutical formulation as claimed in claim 15, wherein the THCV and CBD in the pharmaceutical formulation are in a ratio of from 75:25 to 25:75 THCV:CBD (w/w).

28. A pharmaceutical formulation as claimed in claim 15, wherein the THCV and CBD in the pharmaceutical formulation are in a ratio of from 65:35 to 35:65 THCV:CBD (w/w).

29. A pharmaceutical formulation as claimed in claim 15, wherein the THCV and CBD in the pharmaceutical formulation are in a ratio of from 55:45 to 45:55 THCV:CBD (w/w).

30. A pharmaceutical formulation as claimed in claim 15, wherein the THCV and CBD in the pharmaceutical formulation are in a ratio of approximately 50:50 THCV:CBD (w/w).
